(19) United States
(12) Patent Application Publication
(10) Pub. No.: US 2020/0268633 A1
(43) Pub. Date: Aug. 27, 2020

(54) SKIN TREATMENT

(71) Applicant: Jonathan Joseph Petrillo, Reno, NV (US)

(72) Inventor: Jonathan Joseph Petrillo, Reno, NV (US)

(21) Appl. No.: 16/801,081

(22) Filed: Feb. 25, 2020

Related U.S. Application Data
(60) Provisional application No. 62/810,206, filed on Feb. 25, 2019.

(51) Int. Cl. A61K 8/67; A61K 8/63; A61K 8/60; A61K 8/64

(52) U.S. Cl. CPC A61K 8/676; A61K 8/678; A61K 8/96; A61K 8/602; A61K 8/64; A61K 8/63

(57) ABSTRACT

A composition for use as a skin care formulation may include a combination of Vitamin C (L-ascorbic acid) and pentacyclic triterpene such as Asiatic Acid. A particular formulation may include by weight 5% to 25% Vitamin C (L-ascorbic acid); 0.005% to 2.0% of pentacyclic triterpene such as Asiatic Acid; 5% to 60% of a solvent comprising absolute, denatured alcohol; water. The composition may have a pH no more than about 3.0 and preferably no more than about 2.0. The multi-phase solution composition may also comprise Glutathione, Alpha-Arbutin, and Vitamin E and its derivatives.

### CROSS REFERENCE TO RELATED APPLICATIONS

This application claims priority to U.S. provisional patent application Ser. No. 62/810,206, filed 25 Feb. 2019, the entire contents of which are herein incorporated by reference.

### FIELD OF THE INVENTION

The present invention relates to the treatment of distressed skin exposed to free radical damage and aging skin, in particular through the use of antioxidants.

### BACKGROUND OF THE INVENTION

A free radical is any atom or molecule that has a single unpaired electron in an outer shell as most biologically relevant free radicals are highly reactive. Free radical damage of skin cells is closely associated with oxidative damage causing an imbalance that damages components of cells. Oxidation is a chemical reaction that can produce free radicals, which can lead to chain reactions that may damage the structure of skin cells.

Autoxidation is oxidation that occurs in open air when a substance prone to oxidation becomes in contact with the open air or presence of oxygen.

Antioxidants are reducing agents which limit oxidative damage to structures by passivation, or lessening the affect and breakdown of skin cells from free radicals and the environment. Antioxidants also inhibit oxidation.

What is required is an improved treatment to combat oxidation and free radical processes that affect the skin.

### SUMMARY OF ONE EMBODIMENT OF THE INVENTION

### Advantages of One or More Embodiments of the Present Invention

The various embodiments of the present invention may, but do not necessarily, achieve one or more of the following advantages:

a skin treatment that can treat damaged skin cells;

combat skin aging processes;

provide a low pH formulation.

These and other advantages may be realized by reference to the remaining portions of the specification, claims, and abstract.

### Brief Description of One Embodiment of the Present Invention

In one aspect of the present invention, there is a provided a treatment for skin cells. The treatment may include a composition containing a combination of antioxidants and solvents which produce a low pH and autoxidation preventative properties against free radicals. The multi-phase solution compositions may comprise by weight 5% to 25% Vitamin C (L-ascorbic acid); 0.005% to 2.0% of pentacyclic triterpene such as Asiatic Acid; 5% to 60% of a solvent comprising absolute, denatured alcohol; water; the composition having a pH no more than about 3.0.

In one embodiment, the composition may be a multi-phase solution.

In one embodiment, the composition has a pH of no more than about 2.0.

In a preferred embodiment, the composition may also comprise Glutathione, Alpha-Arbutin, and Vitamin E and/or its derivatives.

The above description sets forth, rather broadly, a summary of one embodiment of the present invention so that the detailed description that follows may be better understood and contributions of the present invention to the art may be better appreciated. Some of the embodiments of the present invention may not include all of the features or characteristics listed in the above summary. There are, of course, additional features of the invention that will be described below and will form the subject matter of claims. In this respect, before explaining at least one preferred embodiment of the invention in detail, it is to be understood that the invention is not limited in its application to the details of the construction and to the arrangement of the components set forth in the following description or as illustrated in the drawings. The invention is capable of other embodiments and of being practiced and carried out in various ways. Also, it is to be understood that the phraseology and terminology employed herein are for the purpose of description and should not be regarded as limiting.

### DESCRIPTION OF CERTAIN EMBODIMENTS OF THE PRESENT INVENTION

In the following detailed description of the preferred embodiments, reference is made to the accompanying drawings, which form a part of this application. The drawings show, by way of illustration, specific embodiments in which the invention may be practiced. It is to be understood that other embodiments may be utilized and structural changes may be made without departing from the scope of the present invention.

The present inventions show that autoxidation is reduced when the skin is treated with a unique combination of antioxidants, thus reducing free radical assault to skin cells. Antioxidants such as Vitamin C (L-ascorbic acid), Asiatic Acid, Glutathione, Vitamin E, and Alpha-Arbutin inhibit oxidation by terminating chain reactions in the prevention of free radical formation. In one embodiment, a composition for treating skin cells provides a super low pH formulation with autoxidation preventative properties against free radicals and potential wound healing properties.

In one embodiment, the composition provides a highly stabilized antioxidant combination of Vitamin C (L-ascorbic acid), Vitamin E (d-tocopherols), Glutathione, Asiatic Acid, and Alpha-Arbutin. A combination of these may improve the appearance of recently damaged skin and may slow the aging process. The compositions comprise Vitamin C as L-ascorbic acid; Asiatic Acid, which is a pentacyclic triterpene antioxidant isolated from a variety of plants; a solvent comprising absolute alcohol; and water; the composition having a pH of no more than about 2.0. The composition also comprises Vitamin E and or its derivatives and is useful in providing benefits to the skin which include wound healing benefits of the skin. The low pH solution facilitates increased absorption of antioxidants, autoxidation preventative properties, anti-inflammatory and neuroprotective properties, particularly to the skin of a subject. The composition also comprises Alpha-Arbutin, preventing the formation of melanin.

A particular formulation may include by weight 5% to 25% Vitamin C (L-ascorbic acid); 0.005% to 2.0% of pentacyclic triterpene such as Asiatic Acid; 5% to 60% of a solvent comprising absolute, denatured alcohol; water. The composition may have a pH no more than about 3.0. The multi-phase solution composition may also comprise Glutathione, Alpha-Arbutin, and Vitamin E and its derivatives.

In one embodiment, the composition has a pH of no more than about 2.5.

In one embodiment, the composition has a pH of no more than about 2.0.

In one embodiment, the Vitamin C component may be in an amount of 10% to 20% by weight. In one embodiment, the Vitamin C component may be approximately 15%. Vitamin C, or L-ascorbic acid can be obtained from any number of proprietors, such as Sigma-Aldrich.

In one embodiment, the pentacyclic triterpene may be in an amount of 0.007% to 1.5% by weight. In one embodiment, the pentacyclic triterpene may in an amount of approximately 0.01%.

Vitamin E may be included in the formulation in any of a variety of forms, including alpha, beta, delta, and gamma tocopherols, and alpha, beta, delta and gamma tocotrienols. The person skilled in the art will readily understand that combinations and/or derivates of these forms may be utilized.

Methods for preparing formulations using L-ascorbic acid are known in the art and no special method or techniques are required to prepare the formulation as described herein.

In a specific embodiment, the formulation may be as follows:
Deionized water—64.07%
Alcohol Denatured—25.0%
1,3-Dihydroxybutane—3.0%
1, 5-Pentanediol—3.0%
Glycerin—2.5%
Zinc Sulfate, Heptahydrate—1.0%
Ferulic Acid—0.1%
Asiatic Acid—0.01%
Citric Acid—0.1%
Hydroxyethylcellulose—0.4%
Potassium Sorbate—0.2%
Sodium Benzoate—0.5%
Xanthan Gum—0.1%
Fragrance—0.02%

1,3-Dihydroxybutane may also be referred to as Butylene Glycol. 1,5-Pentanediol may also be referred to as Pentylene Glycol.

Triethylene Glycol Monododecyl Ether, C12E3; Dodecyl Triethylene Glycol Ether; Laureth-3; Polyoxyethylene 3 Lauryl Ether;

Rosmarinic Acid=3,4-Dihydroxycinnamic Acid (R)-1-Carboxy-2-(3,4-dihydroxyphenyl)ethyl Ester; (R)—O-(3,4-Dihydroxycinnamoyl)-3-(3,4-dihydroxyphenyl)lactic Acid.

Other Means of Identification Synonyms

1,3-BUTANEDIOL: Butane-1,3-diol, beta-Butylene glycol 1,3-Butylene glycol, 1,3-Dihydroxybutane, Methyltrimethylene glycol.

In an alternative embodiment, the formulation may be as follows:
Water—59.19%
Trisodium EDTA—0.10%
Hydroxyethylcellulose—0.30%
Hyaluronic acid—0.01%
Aloe Barbadensis Juice (Aloe Vera juice)—4.00%
Propanediol—13.00%
Glycerin—0.10%
Butylene Glycol—0.10%
Zinc Sulfate—1.00%
Alpha-Arbutin—0.10%
Bioflavonoids—0.01%
L-Glutathione—0.01%
Sorbitol—0.10%
Allantoin—0.10%
Citric Acid—0.10%
Sodium Citrate—0.07%
Vitamin C (L-Ascorbic Acid)—15.00%
Lexgard OE70—0.40%
Phenoxyethanol—0.40%
Asiatic Acid—0.01%
Alcohol Denatured—4.00%
Polysorbate 20—1.50%
Vitamin E (d-toccopherols)—0.10%
NaOH 50% Solution—0.30%

The hydroxyethylcellulose may increase the viscosity of the formulation. The person skilled in the art would readily understand that substitutes may be made for performing a similar function. The Aloe Vera juice, propanediol, sorbitol and allantoin may be optional and may be used to reduce irritation. Alternative ingredients for reducing irritation may be substituted, as would be apparent to the person skilled in the art. An additional benefit of the propanediol is that it may be stabilizing and moisturizing.

While examples of specific formulations are provided herein, the person skilled in the art will readily understand that cosmetic formulations and skin treatments may be enhanced by additional elements such as colorants, dyes, perfumes, deodorants, UV protection elements, etc.

Although the description above contains many specifications, these should not be construed as limiting the scope of the invention but as merely providing illustrations of some of the embodiments of this invention. Thus, the scope of the invention should be determined by the appended claims and their legal equivalents rather than by the examples given.

What is claimed is:

1. A composition comprising by weight 5% to 25% Vitamin C (L-ascorbic acid); 0.005% to 2.0% of pentacyclic triterpene; 5% to 60% of a solvent comprising absolute, denatured alcohol; water; wherein the composition has a pH of no more than about 3.0.

2. The composition of claim 1 comprising Glutathione.

3. The composition of claim 1 comprising Alpha-Arbutin

4. The composition of claim 1 comprising and at least one of Vitamin E or at least one derivative of Vitamin E.

5. The composition of claim 1 wherein the pentacyclic triterpene comprises Asiatic Acid.

6. The composition of claim 1 comprising at least one of Aloe Vera juice, propanediol, sorbitol and allantoin.

7. The composition of claim 1 wherein the composition is a multi-phase composition.

8. The composition of claim 1 wherein the Vitamin C component may be in an amount of 10% to 20% by weight.

9. The composition of claim 1 wherein the Vitamin C component is approximately 15%.

10. The composition of claim 1 wherein the pentacyclic triterpene is in an amount of 0.007% to 1.5% by weight.

11. The composition of claim 1 wherein the pentacyclic triterpene is in an amount of approximately 0.01%.

12. The composition of claim 1 wherein the composition has a pH of no more than about 2.5

13. The composition of claim 1 wherein the composition has a pH of no more than about 2.0
